(19) United States
(12) Patent Application Publication
(10) Pub. No.: US 2020/0368360 A1
(43) Pub. Date: Nov. 26, 2020

(54) SKIN PERMEATION ENHANCING COMPOSITION

(71) Applicant: Professional Compounding Centers of America, Inc., Houston, TX (US)

(72) Inventor: DANIEL BANOV, Sugar Land, TX (US)

(21) Appl. No.: 16/883,625

(22) Filed: May 26, 2020

Related U.S. Application Data
(60) Provisional application No. 62/852,133, filed on May 23, 2019.

(51) Int. Cl. A61K 47/46; A61K 9/06; A61K 9/107; A61K 31/57

(52) U.S. Cl. CPC A61K 47/46; A61K 31/57; A61K 9/107; A61K 9/06

(57) ABSTRACT

A composition for transdermal delivery of pharmaceuticals including a permeation enhancer comprising at least one jojoba constituent, an active pharmaceutical ingredient, and an excipient base.

### CROSS-REFERENCE TO RELATED CASES

This application claims the benefit of U.S. provisional patent application Ser. No. 62/852,133, filed on May 23, 2019, and incorporates such provisional application by reference into this disclosure as if fully set out at this point.

### FIELD OF THE INVENTION

This disclosure relates to permeation enhancement compositions in general and, more specifically, to compositions and methods relying on skin permeation enhancement compositions for specific active pharmaceutical ingredients or cosmetic agents.

### BACKGROUND OF THE INVENTION

The skin provides a protective barrier against foreign materials and infection. In mammals this is accomplished by forming a highly insoluble protein and lipid structure on the surface of the corneocytes termed the cornified envelope (CE). The CE is composed of polar lipids, such as ceramides, sterols, and fatty acids, and a complicated network of cross-linked proteins; however, the cytoplasm of stratum corneum cells remains polar and aqueous. The CE is extremely thin (10 microns) but provides a substantial barrier. Because of the accessibility and large area of the skin, it has long been considered a promising route for the administration of drugs, whether dermal, regional, or systemic effects are desired.

A topical route of drug administration is sometimes desirable because the risks and inconvenience of parenteral treatment can be avoided; the variable absorption and metabolism associated with oral treatment can be circumvented; drug administration can be continuous, thereby permitting the use of pharmacologically active agents with short biological half-lives; the gastrointestinal irritation associated with many compounds can be avoided; and cutaneous manifestations of diseases can be treated more effectively than by systemic approaches.

What is needed is a composition and method for addressing the above, and related issues.

### SUMMARY OF THE INVENTION

The invention of the present disclosure, in one aspect thereof, comprises a composition for transdermal delivery including a permeation enhancer comprising at least one jojoba constituent; an active pharmaceutical ingredient; and an excipient base. In various embodiments, wherein the jojoba constituent comprises jojoba ester, jojoba alcohol, and/or isopropyl jojobate. The jojoba constituent may comprise from 1 to 50% of the composition. In other embodiments, the jojoba constituent comprises from 1-10% of the composition, or from 5 to 10% of the composition.

The invention of the present disclosure, in another aspect thereof, comprises a composition for delivery of an active pharmaceutical ingredient and includes a jojoba constituent formulated as skin permeation enhancing ingredient and a suitable base. The jojoba constituent may comprise jojoba ester, comprises jojoba alcohol, and/or isopropyl jojobate. The jojoba constituent may comprise jojoba ester, jojoba alcohol, and isopropyl jojobate at 1-50% volume. The suitable base may comprise a cream emulsion base. The composition itself may further include the active pharmaceutical ingredient.

The invention of the present disclosure, in another aspect thereof, is a composition consisting of jojoba constituents, active pharmaceutical ingredients; and a suitable carrier base. in some embodiments, the jojoba constituents consist of jojoba esters, jojoba alcohols, isopropyl jojobate, and combinations thereof. The jojoba constituents may range from 1-50% of the composition or may range from 5-10% of the composition. In some cases, the suitable carrier base does not contain further penetration enhancing ingredients nor further active pharmaceutical ingredient. In some cases the suitable carrier base consists of VersaBase® cream 30-3641.

### BRIEF DESCRIPTION OF THE DRAWINGS

FIG. 1 is a side view of a Franz diffusion system for testing compositions of the present disclosure.

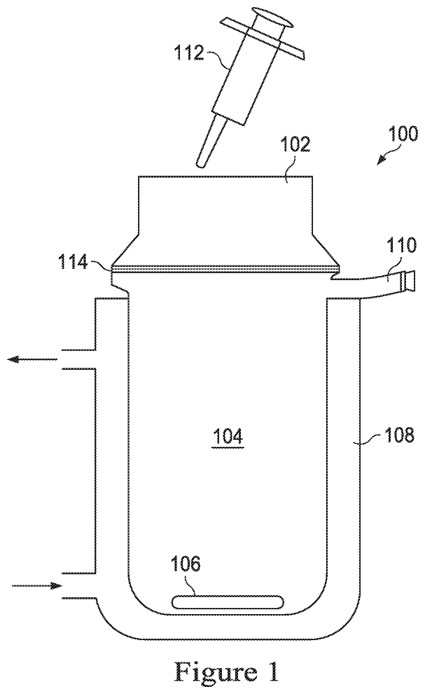

FIG. 2 is a chart illustrating progesterone absorption enhancing performance of a composition according to aspects of the present disclosure.

### DETAILED DESCRIPTION OF THE PREFERRED EMBODIMENTS

In various embodiments, the present disclosure relates to compositions and methods for transdermal and topical drug delivery that can deliver pharmaceuticals and cosmetic agents to skin cells. The present disclosure relates, in some aspects, to enhancement of the penetration of transdermally or topically applied drugs, and to reduction of the skin irritation and other adverse effects that often accompanies transdermally and topically applied actives. The compositions and methods of the present disclosure are also applicable to mucous membrane application in certain embodiments.

Feasibility of the dermal route for systemic drug delivery has been established. Transdermal therapeutic systems include those containing hormones, non-steroidal anti-inflammatory drugs (NSAIDs), glyceryl trinitrate, clonidine, Guaifenesin, nicotine, Ketamine and peptides, and other actives. However, one drawback with drugs applied to the skin or mucous membranes is that they frequently cause irritation. Additionally, many of the necessary components of topical, transdermal, and transmucosal formulations and delivery devices, such as solubilizing agents, suspending agents, dispersing agents, preservatives, animal and vegetable fats, oils, or waxes, stabilizing agents, thickening or gelling agents, buffering agents, adhesive agents, and, particularly, penetration enhancing agents, are irritating alone or in combination. However, in the absence of penetration enhancing agents, few drugs are capable of penetrating the skin or mucous membranes in therapeutically effective concentrations.

Penetration enhancing compounds, processes, or devices can suitably increase drug penetration. Included among these are chemical enhancers, iontophoresis, sonophoresis, and various delivery devices. Penetration enhancement usually results in unwanted irritation. Many transdermal and topical products show high incidences of adverse skin reactions such as scaling, pruritic erythema and allergic contact dermatitis. This is particularly true for topical and transdermal testosterone products currently on the market.

In addition to irritation, some penetration enhancers are regarded as toxic; some irritate the skin; some have a thinning effect on the skin after prolonged use; some change the intactness of the skin structure resulting in a change in the diffusability of the drug. On the other hand, the embodiments of the present disclosure provide for safe and effective transdermal delivery systems that can administer a wide-range of pharmaceuticals and cosmetic agents.

According to embodiments of the present disclosure, transdermal delivery systems or compositions comprise a jojoba derivative such as jojoba esters, jojoba alcohols, and isopropyl jojobate. Jojoba is a shrub that is endemic to North America in the southwest United States, scientific name, Simmondsia chinensis. The shrub's foliage provides food for wildlife and livestock where it is found. Jojoba is cultivated for the oil that may be obtained from its seeds.

Jojoba esters are obtained by hydrogenation or transesterification of jojoba oil. There is no triglyceride component of jojoba esters and thus it may be considered a wax. The esters comprise a mixture of long chain fatty acids and alcohols. The esters may appear as an odorless and colorless liquid, a white cream, or a white wax and are resistant to oxidation. Jojoba alcohol is the alcohol component resulting from saponification of jojoba oil or jojoba esters. It contains saturated and unsaturated alcohol components and it a clear, colorless liquid at room temperature. Isopropyl jojobate is the isopropyl ester of unfractionated saponification of jojoba oil. It may contain mixture of jojoba alcohols and the esters of isopropyl alcohol and jojoba acids. It may appear as an odorless pale liquid with a lower viscosity than jojoba oil. All of these (jojoba esters, jojoba alcohols, and/or isopropyl jojobate) may be considered j oj oba constituents according to the present disclosure. In some formulations, these are the only jojoba-derived ingredients in the compositions. The compositions comprising or consisting of these jojoba constituents may be used as penetration enhancers according to the present disclosure. In some embodiments, only one of the jojoba constituents identified may be used as a penetration enhancing component. In other embodiments, mixture of the jojoba constituents are contemplated.

Some formulations of the present disclosure may be used to deliver pharmaceuticals, therapeutic compounds, and cosmetic ingredients. In some embodiments, a transdermal delivery system comprises a formulation of penetration enhancer (an Isopropyl Jojobate, Jojoba Ester, etc.) that delivers a wide range of pharmaceuticals and cosmetic agents having molecular weights of less than 100 daltons to greater than 500,000 daltons. For example, embodiments of the transdermal delivery system include formulations that deliver a therapeutically effective amount of non-steroidal anti-inflammatory drugs (NSAIDs), low and high molecular weight peptides (e.g., collagens or fragments thereof), hormones, nucleic acids, antibiotics, vaccine preparations, and immunogenic preparations.

Some transdermal delivery system formulations comprise a penetration enhancer including a jojoba ester lipid (e.g., an Isopropyl Jojobate), and a delivered agent (e.g., an amino acid, peptide, nucleic acid, protein, hydrolyzed protein, hormone, chemical, anti-inflammatory or drug). A vegetable oil and/or water and/or an aqueous adjuvant can be mixed with the penetration enhancer to improve the solubility and/or transport of a particular delivered agent.

In some embodiments, a composition comprises about 1% to 50% by weight or volume isopropyl jojobate and jojoba ester mixture lipid and about 1% to 30% by weight water.

In some embodiments phospholipids may be part of the formulation. In some embodiments, about 1% to 25% by weight or volume is isopropyl jojobate and jojoba ester mixture lipid. In further embodiments, a composition comprises 5% to 10% of isopropyl jojobate, jojoba alcohol, and jojoba esters. In some embodiments the jojoba constituent comprises from about 1-5%; 1-10%; 1-50%; 5-10%; 5-50%; or 10-50% weight/weight or volume/volume of the final composition that also includes active ingredients and any bases or excipients. In various embodiments this jojoba constituent may comprise one or more of jojoba esters, jojoba alcohols, and isopropyl jojobate. In some embodiments, the jojoba constituents consist exclusively of these ingredients. In further embodiments, the jojoba constituent consists of any of these exclusively, at the stated amount ranges of the total composition.

According to various embodiments, a composition for topical application having penetration-enhancing properties comprises isopropyl jojobate, jojoba alcohol, and/or jojoba esters. A topical delivery system may additionally comprise anhydrous silicone gel, an emulsion (water/oil; oil/water or oil/water/silicone), solutions, shampoo, or conditioners.

In some embodiments, active agents may comprise about 0.1% to about 30% of the composition w/w or v/v. These may include, but are not limited to, hormones, NSAIDs, cannabinoids, anti-depressives, peptides, growth factors, anti-aging compounds, skin brighteners, scars and wound treatments, and/or others.

In various embodiments of a transdermal delivery system, silicone gels or anhydrous silicone can be part of a composition. The composition or formulation may be used in water containing products such as emulsions, in which water and phospholipids may be present. In various embodiments, the transdermal delivery systems described herein can also include fragrances, creams, bases and other ingredients that stabilize the formulation, facilitate delivery, or protect the delivered agent from degradation (e.g., agents that inhibit DNAse, RNAse, or proteases).

Embodiment of the present disclosure may be applied or delivered according to various methods. For example, a composition according to the present disclosure may be placed into a vessel that is joined to an applicator such that the active ingredients can be easily provided to a subject. Applicators include, but are not limited to, roll-ons, bottles, jars, tubes, sprayer, atomizers, brushes, swabs, gel dispensing devices, and other dispensing devices.

Various methods of treatment and modes of delivery or application are also contemplated. For example, one method includes of reducing pain or inflammation by using a transdermal delivery system that comprises an anti-inflammatory molecule (e.g., an NSAID or MSM) on a subject in need of a reduction of pain or inflammation. Monitoring the reduction in inflammation may also be desired as part of a rehabilitation program. Some formulations of transdermal delivery systems of the present disclosure can be used to reduce oxidative stress to cells, tissues and the body of a subject.

Other formulations of a transdermal delivery system of the present disclosure may be used to reduce psoriasis, eczema, or related conditions. Some embodiments may be used to promote wound healing in a subject in need thereof. By one approach, a transdermal delivery system that comprises peptides that promote wound healing are provided to a subject in need of a treatment or reduction in psoriasis or eczema or a condition associated with psoriasis, eczema (e.g., allergies), or treatment of a wound.

Other formulations of a transdermal delivery system of the present disclosure may be used to relax the muscles of a subject. By one approach, a transdermal delivery system that comprises a compound that relaxes the muscles (e.g., Ketoprofen, Guaifenesin or ibuprofen) and is provided to a subject in need of a muscle relaxant. Accordingly, methods of treating or preventing muscle soreness are embodiments of the present disclosure.

Embodiments of the present disclosure comprise a method of increasing skin or mucus membrane penetration utilizing a permeation enhancer according to the formulations described herein (e.g., including isopropyl jojobate, jojoba alcohol, and/or jojoba esters) for delivery of a hormone molecule (e.g., estrogen or androgens, derivatives or functional analogues thereof) on a subject in need of an increased hormone level. Accordingly, methods of treating or preventing a hormone deficiency or methods of increasing the level of a hormone in a subject using one of the transdermal delivery systems described herein are contemplated embodiments.

Some embodiments comprise a method of increasing penetration using a transdermal delivery topical system according to the formulations described herein for delivery of cannabinoids such as CBD and THC and hemp oil. Some embodiments comprise a method of increasing penetration using a transdermal delivery topical system according to the formulations described herein for delivery anti-inflammatory molecule (e.g., an NSAID or MSM) on a subject in need of a reduction of pain or inflammation.

In some embodiments, formulations of transdermal delivery system can be used to relax the muscles of a subject. By one approach, a transdermal delivery system that comprises a compound that relaxes the muscles (e.g., ketamine, Ketoprofen, Guaifenesin or ibuprofen) is provided to a subject in need of a muscle relaxant along with a formulation as described herein to increase permeation. According to some embodiments of the transdermal delivery system described herein is used to brighten the skin, reduce age spots or skin discolorations, reduce stretch marks, reduce spider veins, or add dyes, inks, (e.g., tattoo ink), or apply perfumes or fragrances to the skin of a subject.

It should be understood that formulations of the present embodiment may include excipients or bases as well as the penetration enhancing components and/or active ingredients intended to treat a specific condition or have a specific desired effect or use. In various embodiments, excipient compositions as are known in the art can be used to stabilize, carry, and/or apply the penetration enhancer and active ingredients. The excipient should not contain any substances that unacceptably degrade, counter act, otherwise unacceptably alter the penetration enhancer or active ingredient. One excipient or base product that works well with the penetration enhancing compositions and active ingredients of the present application is VersaBase® 30-3641 available from PCCA of Houston, Tex. Other VersaBase® products such as gel (30-3656), lotion (30-3653), or shampoo (30-4195) may be used in some embodiments.

In some embodiments, compositions include only the base, penetration enhancer, and one or more active ingredients. In other embodiments, other components may be present. In some embodiments, the permeation enhancer comprises from 0-99% w/w or v/v of a composition. Compositions according to the present disclosure may be prepared in a cream base emulsion at various percent by weight or volume of the permeation enhancer or the entire composition including active pharmaceuticals.

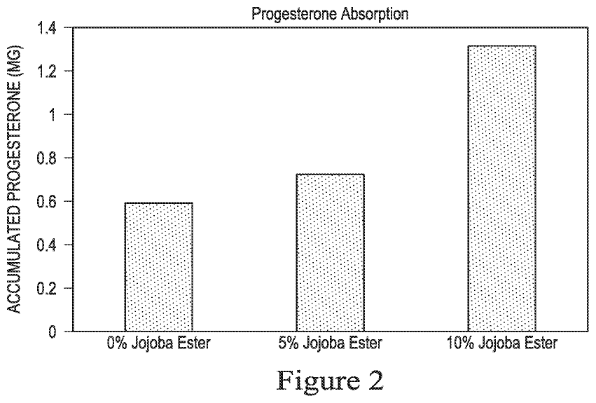

With reference now to FIG. 2, a testing apparatus 100 is shown, wherein was conducted a further test of the compositions according to the present disclosure. An exemplary Franz diffusion system 100 was utilized to test absorption of progesterone with varying concentrations of jojoba ester as a jojoba constituent penetration enhancer. In all cases the progesterone concentration was 10%. One test was conducted with a concentration of 0% jojoba ester, another with 5% jojoba ester, and another with 10% jojoba ester. The remainder of the formulation consisted of VersaBase® in all cases.

The Franz diffusion system 100 included a Strat-M Membrane 114 (from EMD Millipore Corporation) having a surface area of 1.77 cm². A receptor medium (HBSS 14175-079, 25 mM HEPES, 15630-080 and 50 μg/mL Gentamicin, 15750-060, Gibco) was added to a receptor compartment 104. Approximately 400 mg of test product was applied in each case to a donor compartment 102 using a positive displacement pipette 112. The receptor medium solution was stirred magnetically at approximately ˜600 RPM with a magnetic stirrer 106 with a water jacket 108 temperature maintained at 32±0.5° C.

During an exposure period of each run, samples of the receptor solutions (1 mL) were removed (e.g., via port 110) at pre-selected times, here 8 and 24 hours. The collected 8-hour samples were then concentrated and the corresponding residue was reconstituted with methanol.

The quantification of progesterone was performed by UPLC (Ultra Performance Liquid Chromatography), using a reverse phase, gradient chromatography and the following mobile phases: A (Purified water); B (0.1% formic acid in acetonitrile). The detector used was an ultraviolet photodiode array with a detection wavelength of 244 nanometers. All samples were filtered with a PVDF membrane before being injected into the UPLC.

The total absorption of progesterone (i.e., the total recovered in the receptor solutions over 24 hours from a single application) was 0.591 μg for progesterone at 10% with 0% Jojoba Ester in VersaBase, 0.724 μg for Progesterone at 10% with 5% Jojoba Ester in VersaBase, and 1.316 μg for Progesterone at 10% with 0% Jojoba Ester in VersaBase.

The results are summarized Table A below. The results indicate that, compared to a formulation of progesterone without jojoba ester, the absorption of progesterone for formulations of progesterone with 5% jojoba ester and progesterone with 10% jojoba ester increased 122.5% and 222.7%, respectively. FIG. 3 illustrates the results graphically.

TABLE A
Absorption of progesterone with or
without jojoba ester over 24 hours.

Accumulated Amount

| Formulation | μg | % |
|---|---|---|
| Progesterone 10% with 0% Jojoba | 0.591 | 100% |
| Ester in VersaBase | | |
| Progesterone 10% with 5% Jojoba | 0.724 | 122.50% |
| Ester in VersaBase | | |
| Progesterone 10% with 10% Jojoba | 1.316 | 222.70% |
| Ester in VersaBase | | |

It is to be understood that the terms “including” and “comprising” and grammatical variants thereof do not preclude the addition of one or more components, features, steps, or integers or groups thereof and that the terms are to be construed as specifying components, features, steps or integers. Use of the term, “consisting” or “consisting of” is intended to preclude other steps, components, ingredients, etc. within the context of the specific step, component, etc. identified.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26 -100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A composition for transdermal delivery comprising:
a permeation enhancer comprising at least one jojoba constituent;
an active pharmaceutical ingredient; and
an excipient base.

2. The composition of claim 1, wherein the jojoba constituent comprises jojoba ester.

3. The composition of claim 1, wherein the jojoba constituent comprises jojoba alcohol.

4. The composition of claim 1, wherein the jojoba constituent comprises isopropyl jojobate.

5. The composition of claim 1, wherein the jojoba constituent comprises from 1 to 50% of the composition.

6. The composition of claim 1, wherein the jojoba constituent comprises from 1-10% of the composition.

7. The composition of claim 1, wherein the jojoba constituent comprises from 5 to 10% of the composition.

8. A composition for delivery of an active pharmaceutical ingredient comprising:
a jojoba constituent formulated as skin permeation enhancing ingredient; and
a suitable base.

9. The composition of claim 8, wherein the jojoba constituent comprises jojoba ester.

10. The composition of claim 9, wherein the jojoba constituent comprises jojoba alcohol.

11. The composition of claim 10, wherein the jojoba constituent comprises isopropyl jojobate.

12. The composition of claim 8, wherein the jojoba constituent comprises jojoba ester, jojoba alcohol, and isopropyl jojobate at 1-50% volume.

13. The composition of claim 12, wherein the suitable base comprises a cream emulsion base.

14. The composition of claim 13, further comprising the active pharmaceutical ingredient.

15. A composition consisting of:
jojoba constituents;
active pharmaceutical ingredients; and
a suitable carrier base.

16. The composition of claim 15, wherein the jojoba constituents consist of jojoba esters, jojoba alcohols, isopropyl jojobate, and combinations thereof.

17. The composition of claim 16, wherein the jojoba constituents are from 1-50% of the composition.

18. The composition of claim 17, wherein the jojoba constituents are from 5-10% of the composition.

19. The composition of claim 18, wherein the suitable carrier base does not contain further penetration enhancing ingredients nor further active pharmaceutical ingredient.

20. The composition of claim 18, wherein the suitable carrier base consists of VersaBase® cream 30-3641.
